(19) United States
(12) Patent Application Publication
(10) Pub. No.: US 2023/0304037 A1
(43) Pub. Date: Sep. 28, 2023

(54) METHODS FOR PRODUCING CLINICAL-GRADE LENTIVIRAL VECTOR

(71) Applicant: Tmunity Therapeutics Inc., Philadelphia, PA (US)

(72) Inventors: Michael Sean Burnham, Mount Royal, NJ (US); Maryam Rahimian, Dresher, PA (US)

(73) Assignee: Tmunity Therapeutics Inc., Philadelphia, PA (US)

(21) Appl. No.: 18/020,605

(22) PCT Filed: Aug. 12, 2021
(86) PCT No.: PCT/US2021/045677

§ 371 Date: Feb. 9, 2023

Related U.S. Application Data
(60) Provisional application No. 63/065,225, filed on Aug. 13, 2020.

(51) Int. Cl. C12N 15/86; C12N 9/22; C12N 5/0783

(52) U.S. Cl. CPC C12N 15/86; C12N 9/22; C12Y 301/30002; C12N 5/0636; C12N 2740/15041

(57) ABSTRACT

Described are improved methods for manufacturing and purifying clinical-grade retroviral vectors, such as lentiviral vectors.

### CROSS-REFERENCE TO RELATED APPLICATIONS

This application is a PCT application which claims a priority benefit to U.S. Provisional Application No. 63/065,225, filed Aug. 13, 2020; the entirety of which is herein expressly incorporated by reference.

### BACKGROUND

Novel treatments using T-cells engineered to express immune receptors have resulted in promising immunotherapies for a wide swath of intractable diseases, including cancer and autoimmune diseases. Lentiviral vectors have been used for the delivery of transgenes in a number of different applications because lentiviruses are able to infect non-dividing cells (Lewis and Emerman (1993) J. Virol. 68:510). Additionally, lentiviral vectors allow for very stable, long-term expression of the transgene.

The process of producing lentiviral vectors is no simple feat; one that requires numerous steps. While many processes will yield some degree of success, the need for highly pure samples free from all number of biotic and abiotic contaminants for clinical applications of the vectors further complicates these processes. The vector purification process is a critical step in clinical gene transfer therapies, and novel methods are needed to provide viral vectors more efficiently, safely, and at a great scale. The present disclosure addresses this need.

### SUMMARY OF THE DISCLOSURE

The present disclosure is generally drawn to processes for producing and purifying lentiviral vectors.

In some aspects, the disclosure is broadly drawn to a process for producing a lentiviral vector formulation, the process comprising (a) treating a filter-sterilized lentiviral vector preparation with a nuclease, and (b) concentrating the nuclease-treated lentiviral vector preparation to produce the lentiviral vector formulation; wherein the step of concentration is the final step in the process. In some aspects, the process comprises a clarification of cell culture supernatant. In some aspects, the clarification is followed by a first nuclease treatment. In some aspects, the nuclease possesses endonuclease activity. In some aspects, the nuclease possesses exonuclease activity. In some aspects, the nuclease is a modified nuclease from Serratia marcescens.

In some aspects, the first nuclease treatment precedes an ultrafiltration/diafiltration step. In some aspects, the ultrafiltration/diafiltration step comprises tangential flow filtration. In some aspects, the ultrafiltration/diafiltration step comprises hollow fiber filtration.

In some aspects, the lentiviral vector preparation is diluted prior to the filter-sterilization of the lentiviral vector preparation. In some aspects, the lentiviral vector preparation is diluted into a formulation buffer. In some aspects, the lentiviral vector preparation is diluted into the formulation buffer during the ultrafiltration/diafiltration step.

In some aspects, the process comprises a second nuclease treatment. In some aspects, the second nuclease treatment is the penultimate step in the process. In some aspects, the nuclease possesses endonuclease activity. In some aspects, the nuclease possesses exonuclease activity. In some aspects, the nuclease is a modified nuclease from Serratia marcescens.

In some aspects, the disclosure is generally drawn to a process for producing a lentiviral vector formulation, the process comprising: (a) culturing cells that produce the lentiviral vector; (b) collecting supernatant from the cultured cells; (c) clarifying the supernatant; (d) concentrating the clarified supernatant; (e) purifying the lentiviral vector from the concentrated supernatant to produce a lentiviral vector preparation; (f) filter-sterilizing the lentiviral vector preparation; (g) treating the filter-sterilized lentiviral vector preparation with one or more nucleases; and (h) concentrating the nuclease-treated lentiviral vector preparation to produce a final product.

In some aspects, the disclosure is generally drawn to a process for producing a lentiviral vector formulation, the process comprising: (a) culturing cells that produce the lentiviral vector; (b) collecting supernatant from the cultured cells; (c) clarifying the supernatant; (d) concentrating the clarified supernatant (e) purifying the lentiviral vector from the concentrated supernatant to produce a lentiviral vector preparation; (f) filter-sterilizing the lentiviral vector preparation; and (g) concentrating the nuclease-treated lentiviral vector preparation to produce a final product; wherein the clarified supernatant and/or the lentiviral vector preparation are treated with a nuclease. In some aspects, concentrating the clarified supernatant comprises exchanging the concentrated supernatant with a formulation buffer.

In some aspects, the clarified supernatant and the lentiviral vector preparation are treated with a nuclease. In some aspects, the clarified supernatant is treated with the nuclease prior to (d), In some aspects, the clarified supernatant is treated with the nuclease after (c). In some aspects, the nuclease possesses endonuclease activity. In some aspects, the nuclease possesses exonuclease activity. In some aspects, the nuclease is a modified nuclease from Serratia marcescens.

In some aspects, the disclosure is generally drawn to a process for producing a lentiviral vector formulation, comprising in chronological order: (a) culturing cells that produce the lentiviral vector; (b) collecting the supernatant comprising the lentiviral vector; (c) clarifying the supernatant; (d) treating the clarified supernatant with a nuclease; (e) concentrating the nuclease-treated clarified supernatant, comprising exchange of the concentrated supernatant with formulation buffer; (f) purifying the lentiviral vector from the concentrated supernatant to produce a lentiviral preparation; (g) filter-sterilizing the lentiviral vector preparation; (h) treating the filter-sterilized lentiviral vector preparation with a nuclease; and (i) concentrating the nuclease-treated lentiviral vector preparation to produce a final product.

In some aspects, the disclosure is generally drawn to a process for producing a viral vector formulation, comprising: culturing cells that produce the viral vector, collecting supernatant from the cultured cells, clarifying the supernatant, concentrating the clarified supernatant, purifying the lentiviral vector from the concentrated supernatant to produce a viral vector preparation, filter-sterilizing the lentiviral vector preparation, and concentrating the nuclease-treated lentiviral vector preparation to produce a final product; wherein the process preferably includes treating the vectors with only a single nuclease step, and wherein purification of the vector may be (1) via one or more rounds of chromatography, (2) via high speed centrifugation or ultracentrifugation, or (3) via a concentration solution such as PEG or LENTI-X CONCENTRATOR. In some aspects, the process comprises two separate nuclease treatment steps. In some aspects, one of the two separate nuclease treatment steps are replaced by a vector purification step comprising purification of the vector via (1) one or more rounds of chromatography, (2) high speed centrifugation or ultracentrifugation, or (3) a concentration solution such as PEG or LENTI-X CONCENTRATOR. In some aspects, the process described herein comprises two separate nuclease treatment steps, a first nuclease treatment and a second nuclease treatment. In some aspects, the first nuclease treatment is replaced by a vector purification step comprising purification of the vector via (1) one or more rounds of chromatography, (2) high speed centrifugation or ultracentrifugation, or (3) a concentration solution such as PEG or LENTI-X CONCENTRATOR. In some aspects, the second nuclease treatment is replaced by a vector purification step comprising purification of the vector via (1) one or more rounds of chromatography, (2) high speed centrifugation or ultracentrifugation, or (3) a concentration solution such as PEG or LENTI-X CONCENTRATOR.

The inventions described and claimed herein have many attributes and embodiments including, but not limited to, those set forth or described or referenced in this Brief Summary and the following Brief Description of the Drawings and Detailed Description. It is not intended to be all-inclusive and the inventions described and claimed herein are not limited to or by the features or embodiments identified in this Brief Summary, which is included for purposes of illustration only and not restriction. Additional embodiments may be disclosed in the Detailed Description below.

### BRIEF DESCRIPTION OF THE FIGURES

FIG. 1 depicts a schematic of a viral purification process of the present disclosure. The recitation of CC700 corresponds to a column comprising CAPTOCORE 700, a multimodal chromatography resin used for purification/polishing of viruses and other large biomolecules.

FIG. 2 illustrates the results of a qPCR assay in which a vesicular stomatitis virus glycoprotein (VSV-G) gene was detected and quantified. The VSV-G detection effectively measures the amount of residual VSV-G still present at multiple points in the vector manufacturing process. The amount of residual VSV-G is generally a measure of the amount of residual plasmid contaminants left over from production of the vector. The figure is a graphical representation of the step removal percent seen after three steps in the process of producing the vectors. The “Harvest” sample was collected immediately after clarification. The “TFF 1” sample was collected immediately after the first tangential flow filtration step. The “TFF 2” sample was collected immediately after the final tangential flow filtration step, thus yielding the end result of the lentiviral vector production and purification method. The data is normalized such that the amount of VSV-G in the harvest sample is 1, or 100%, and the amount of VSV-G in the TFF1 sample is 0.16 or 16% and the amount of VSV-G in the TFF2 sample is 0.009 or 0.09%.

FIG. 3 illustrates the results of an assay for determining the effect of various lentiviral vector titers on the total percent yield of the lentiviral vectors subjected to viral purification processes of the present disclosure.

### DETAILED DESCRIPTION

### I. Overview

The application is drawn to methods of manufacturing retroviral vectors, such as lentiviral vectors, and formulations comprising these vectors, particularly those that are isolated at high titers and sufficiently free from contamination with cellular debris, nucleic acid degrading molecules, and proteolytic substances. It is no simple task to produce a purified formulation of retroviral vectors, such as lentiviral vectors, in high titers and free of contaminants. The inventors have identified multiple aspects of the retroviral vector purification process which surprisingly result in increasing the titer of the retroviral (e.g., lentiviral) vector stock.

In particular, the present disclosure is directed to two aspects of manufacturing of retroviral (e.g., lentiviral) vectors: (1) dilution of virus stock prior to sterile filtration; and (2) residual and genomic DNA removal in the final vector production. Residual nucleic acids in a final retroviral product are undesirable. The two aspects of the new viral vector manufacturing process described herein improve overall yield as well as producing an improved end product.

The first aspect is drawn to the concentration of retroviral (e.g., lentiviral) vectors, which has an outsized impact on the titer of the vectors that are recovered in the purification process. More dilute samples of retroviral (e.g., lentiviral) vectors exhibit a greater total percent recovery as compared to more concentrated retroviral (e.g., lentiviral) vector samples. It was surprisingly discovered that in one aspect about a 2×10⁶ infectious titer of retroviral (e.g., lentiviral) vector, or a range encompassing this amount of 1.5×10⁶ to 2.5×10⁶, results in the most efficient retroviral (e.g., lentiviral) vector recovery.

The second aspect of the present disclosure is drawn to the efficiency of removing host cell and genomic DNA from retroviral (e.g., lentiviral) vector samples at one of more stages, which also has an outsized impact on the titer of the recovered retroviral (e.g., lentiviral) vectors. Specifically, it was surprisingly discovered that adding at least one nuclease treatment to a diluted vector after a filter sterilization step, but prior to a vector concentrating step (using e.g., tangential flow filtration (TFF)), increased the host cell and genomic DNA removal efficiency. Any suitable nuclease treatment can be used, and exemplary treatments are described herein. In an exemplary aspect, to be able to increase the efficiency of residual nucleic acid removal, the present inventors demonstrated that a molecular size between about 10 kDa to about 1,000 kDa for hollow fiber or cassette TFF can be used to increase the host cell and genomic DNA removal efficiency.

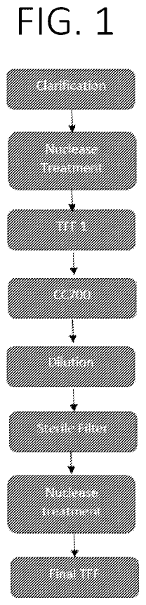

An exemplary flow chart of a lentiviral vector manufacturing process using the novel aspects described herein is shown in FIG. 1.

In an exemplary downstream process described herein, there are two purification steps (e.g., using TFF), and a nuclease treatment step is performed prior to each purification (e.g., TFF) step. The inventors surprisingly demonstrated that additional efficiency increases performing the vector purification methods with two concentration steps, with a nuclease treatment step occurring prior to each.

Furthermore, performing a first nuclease step on a clarified supernatant prior to a first concentration step was found to aid in removing residual DNA and prevent the filter (e.g., TFF) from clogging. However, the second nuclease treatment before a final concentration step removes the residual DNA effectively without any increase in time to the downstream manufacturing steps. The time and temperature of incubation for each nuclease treatment can be different to maximize the process time and efficiency of our purification method.

The inventors demonstrate that a higher molecular weight pore cutoff of about 750 kDa to about 1,000 kDa effectively removes nuclease so there is no concern regarding residual nucleases in the final product.

In a further aspect, the nuclease process is conducted at a reduced temperature, e.g., which is not room temperature or e.g., 30°, which is a typical temperature nuclease processes are conducted at. For example, as further described herein, the nuclease process can be conducted at a “cold” temperature of about 2 to about 8° C. Other exemplary temperatures at which the nuclease process can be conducted are described herein.

The introduction of these additional steps into the lentiviral vector purification process necessarily increases the amount of time it takes to produce viable vectors, adding time to a process that already takes ˜3 days. Adding about an additional day of work to an already time-consuming process would have dissuaded others from pursuing this path.

The vector purification process is a critical step in creating clinical gene transfer therapies, and novel methods are needed to provide viral vectors more efficiently, safely, and at high titer. The present disclosure addresses this need.

### II. Retroviral Vectors

In some aspects, the virus is a retroviral vector. In some aspects, the virus is a recombinant retroviral vector comprising a heterologous transgene or nucleic acid sequence of interest. In some aspects, the heterologous transgene or nucleic acid sequence may be be used in a therapeutic setting for gene therapy purposes. In some aspects, the retroviral vector comprising a heterologous transgene or nucleic acid sequence of interest may be be used for transducing immune cells.

In some aspects, the vector is targeted to a desired cell type. In some aspects, the vector is targeted to a desired cell type to which the vector will fuse with in the process of vector-mediated gene transduction. In further aspects, the desired cell type is an immune cell. In some aspects, the desired cell type is a T-cell, a B-cell, a dendritic cell, or an antigen presenting cell.

In some aspects, the heterologous transgene or nucleic acid sequence of interest may have a therapeutic or diagnostic application. Suitable transgenes or nucleic acid sequences of interest may include, but are not limited to sequences encoding enzymes, cytokines, chemokines, hormones, antibodies, and antioxidant molecules. In some aspects, the transgene or nucleic acid sequences of interest may include engineered immunoglobulin-like molecules, immunomodulatory molecules, anti-sense RNA, microRNA, shRNA, siRNA, ribozymes, gene editing systems (e.g., CRISPR/Cas, zinc-finger nucleases, TALENs), antigen receptors (e.g., chimeric antigen receptors, T cell receptors), an antigen, a toxin, a transdomain negative mutant of a target protein, a tumor suppressor protein, growth factors, membrane proteins, reporter proteins (e.g., fluorescent proteins), and derivatives thereof

In some aspects, the retroviral vector is a lentiviral vector. In some aspects, the lentiviral vector is from bovine immunodeficiency virus, caprine arthritis encephalitis virus, equine infections anemia virus, feline immunodeficiency virus, Human immunodeficiency virus, human immunodeficiency virus 1, human immunodeficiency virus 2, jembrana disease virus, pluma lentivirus, simian immunodeficiency virus, or visna-maedi virus.

In some aspects, the lentiviral vector is pseudotyped, meaning that it comprises an envelope glycoprotein derived from a different virus. In some aspects, the lentiviral vector is pseudotyped with an envelope glycoprotein derived from vesicular stomatitis virus (VSV-G), measles virus, modified measles virus, baboon endogenous virus, gibbon ape leukemia virus. In some aspects, the lentiviral vector is pseudotyped with a modified enveloped glycoprotein. In some aspects, the lentiviral vector is pseudotyped with a chimeric envelope glycoprotein.

In some aspects, the lentiviral vector is modified such that one or more protein coding regions necessary for replication are removed from the lentiviral vector, thus rendering the vector replication deficient. In some aspects, the heterologous transgene or nucleic acid sequence of interest displaces or replaces a portion of the viral genome. In some aspects, the heterologous transgene or nucleic acid sequence of interest may be added to the viral genome thus rendering the vector replication sufficient. In some aspects, the vectors are non-integrating vectors, as described in U.S. Pat. Appln. Pub. No. US20090014754A1. In some aspects, the lentiviral vector is modified such that one or more protein coding regions necessary for replication are removed from the lentiviral vector, as described in U.S. Pat. Appln. Pub. No. US20090075370A1.

### III. Retroviral Vector Production Systems

Retroviral vectors can be propagated in producer or packaging cells. A producer or packaging cell can be any cell wherein a retroviral vector can be propagated, and subsequently harvested. In some aspects, the producer or packaging cell can be a stable producer or host cell line to propagate quantities of viral particles for subsequent purification. In some aspects, the producer or packaging cell is selected from a human cell. In further aspects, the human cell is HEK293, HEK293T, HEK293FT, Te671, HT1080, or CEM. In some aspects, the produce or packaging cell is selected from a murine cell. In further aspects, the murine cell is NIH-3T3. In some aspects, the producer or packaging cell is selected from a mustelidae cell. In further aspects, the mustelidae cell is Mpf. In some aspects, the producer or packaging cell is selected from a canine cell. In further aspects, the canine cell is D17. In some aspects, the producer or packaging cell is a HEK293 cell. In some aspects, transient transfection can be used to generate the lentiviral vector in producer and/or packaging cells.

The terms “packaging cell” and “producer cell” as used herein, refer to a cell which contains the elements necessary for the production of a recombinant retroviral or lentiviral virus, which is lacking in the viral genome. Typically, such packaging cells contain one or more producer plasmids which are capable of expressing viral structural proteins (such as codon optimized gag-pol and env) but do not contain a packaging signal. Preferably, producer/packaging cells are derived from a mammalian cell, and preferably, from a primate cell such as a human cell. In some aspects, the human cell is a human embryonic kidney (HEK) cell. Any type of cell that is capable of supporting replication of a recombinant retroviral or lentiviral virus may be used to propagate the recombinant viruses.

In some aspects, the packaging/producer cell lines have been modified wherein the 3′LTR of the provirus is deleted to improve safety. Further improvements have been made to introduce the gag-pol and env genes on separate plasmids, and can further be introduced into the cell line sequentially to avoid recombination. In some aspects, the recombinant virus and transgene are introduced into the packaging/producer cell line as a third-generation lentivirus system. This lentivirus system is introduced into the cell as four separate plasmids: a plasmid encoding gag-pol, a plasmid encoding the viral rev gene, an envelope plasmid encoding the envelope glycoprotein (e.g., VSV-G), and a transfer plasmid encoding the transgene or the nucleic acid sequence of interest.

Cells transfected with the retroviral vector system are cultured to increase cell and virus numbers and/or virus titer by means and methods well known to persons skilled in the art, and includes but is not limited to providing the proper nutrients for the cell in the appropriate culture media. The methods may comprise growth adhering to surfaces, growth in suspension, or combinations thereof. Culturing can be done for instance in tissue culture flasks, dishes, roller bottles or in bioreactors, using batch, fed-batch, continuous systems, hollow fiber, and the like, hi order to achieve large scale (continuous) production of virus through cell culture it is preferred in the art to have cells capable of growing in suspension. Suitable conditions for culturing cells are known (see e.g. Tissue Culture, Academic Press, Kruse and Paterson, editors (1973), and R. I. Freshney, Culture of animal cells: A manual of basic technique, fourth edition (Wiley-Liss Inc., 2000, ISBN 0-471-34889-9).

In some aspects, the cells are grown in tissue culture flasks and subsequently grown in multilayered culture chambers to generate the recombinant viral particle producing cells. In some aspects, the producer cells are adherent cells to propagate the viral particles. In some aspects, the producer cells are non-adherent cells to propagate the viral particles.

In some aspects, the cells are grown in a medium adequately suited for cultivation of the chosen cell-type and for producing the lentiviral vector. In some aspects, the medium is a complex medium or a minimal medium. In some aspects, the medium is supplemented with antibiotics, mammalian blood serum (such as fetal bovine serum), a pH indicator, etc. In some aspects, the medium is a serum-free medium.

### IV. Purification of Retroviral Vectors

An exemplary vector purification process is exemplified in FIG. 1. In some aspects, the purification process begins with clarifying the spent cell culture medium/supernatant. In further aspects, clarification is followed by (1) one or more concentration steps, (2) one or more nuclease treatment steps, (3) one or more purification steps, and (4) one or more sterile filtration steps. In further aspects, clarification is followed by one or two (or more) concentration steps, (2) one or two (or more) nuclease treatment steps, (3) one or two (or more) purification steps, and (4) one or two (or more) sterile filtration steps; wherein the steps may or may not be performed in stepwise order from (1)-(4). In further aspects, clarification is followed by one or more dilution steps, which may or may not be performed immediately after clarification and before a subsequent step.

In some aspects, the purification process comprises or consists of, in chronological order, (1) clarifying the spent culture medium/supernatant, (2) a first concentration step, (3) a nuclease step, (4) a purification step, (5) a sterile filtration step, and (6) and a second concentration step. In some aspects, the purification process comprises or consists of, in chronological order, (1) clarifying the spent culture medium/supernatant, (2) a nuclease step, (3) a first concentration step, (4) a purification step, (5) a sterile filtration step, and (6) and a second concentration step. In some aspects, the purification process comprises or consists of, in chronological order, (1) clarifying the spent culture medium/supernatant, (2) a first nuclease step, (3) a first concentration step, (4) a purification step, (5) a sterile filtration step, (6) a second nuclease step, and (7) a second concentration step. In some aspects, the purification process may comprise one or more dilution steps.

In some aspects, the first nuclease treatment occurs before the first concentration step. In some aspects, the second nuclease treatment occurs before the second concentration step. In some aspects, the first and second nuclease steps occur before the final concentration step. In some aspects, the first and second nuclease steps do not occur consecutively. In some aspects, the first and second purification steps do not occur consecutively. In some aspects, the first and second purification steps do not occur consecutively. In some aspects, the first and second purification steps do not occur consecutively. In some aspects, the first and second concentration steps do not occur simultaneously. In some aspects, the first and second nuclease steps do not occur consecutively. In some aspects, the first and second purification steps do not occur consecutively.

In some aspects, a concentration step is the final step in the process. In some aspects, if a first and second nuclease step occurs, the first nuclease step occurs before the first concentration step and the second nuclease step occurs before the second concentration step. In some aspects, the dilution step occurs after either the first nuclease step or after both nuclease steps. In some aspects, a nuclease step may occur before the clarification step. In some aspects, the sterile filtration step and the concentration step occur in a closed system such that the filtrate flows from the sterile filter directly to the concentration device.

In some aspects, the sterile filtration step is not the final step of the purification process. In some aspects, the sterile filtration step is not the penultimate step of the purification process. In some aspects, the second nuclease treatment step does not occur before the sterile filtration step. In some aspects, when the sterile filtration step is the final step in the purification process, the vector yield decreases, relative to a control in which the sterile filtration step does not occur last or relative to the stepwise convention set forth in FIG. 1.

In some aspects, the concentration step, is an ultrafiltration step, sometimes referred to as diafiltration when used for buffer exchange. In some aspects, the ultrafiltration/diafiltration step concentrates the vector. In some aspects, the ultrafiltration/diafiltration may be in the form tangential flow filtration (TFF). In some aspects, the ultrafiltration/diafiltration membrane will be selected to have a pore size sufficiently small enough to retain the vector, and sufficiently large enough to effectively clear impurities.

Over the course of culturing the cells transfected with the lentiviral vector, the lentiviral vector the lentiviral vector accumulates in the spent culture medium in which the cells comprising the culture medium are cultivated in. In some aspects, any remaining intact cells in the culture medium are lysed, thus freeing the remaining lentiviral vectors.

In some aspects, the spent medium/cell culture supernatant is clarified. Clarification is the removal of cellular debris from the supernatant as a means to begin isolating the lentiviral vectors.

In some aspects, clarification of the cell culture supernatant is performed by a filtration step to remove cell debris and other impurities. Suitable filters may utilize cellulose filters, regenerated cellulose fibers, cellulose fibers combined with inorganic filter aids (e.g. diatomaceous earth, perlite, fumed silica), cellulose filters combined with inorganic filter aids and organic resins, or any combination thereof, and polymeric filters (examples include but are not limited to nylon, polypropylene, polyethersulfone) to achieve effective removal and acceptable recoveries. In general, a multiple stage process is preferable but not required. An exemplary two or three-stage process would consist of a coarse filter(s) to remove large precipitate and cell debris followed by polishing second stage filter(s) with nominal pore sizes greater than 0.2 micron but less than 1 micron. The optimal combination may be a function of the precipitate size distribution as well as other variables. In addition, single stage operations employing a relatively small pore size filter or centrifugation may also be used for clarification. More generally, any clarification approach including but not limited to dead-end filtration, microfiltration, centrifugation, or body feed of filter aids (e.g. diatomaceous earth) in combination with dead-end or depth filtration, which provides a filtrate of suitable clarity to not foul the membrane and/or resins in the subsequent steps, will be acceptable to use in the clarification step of the present invention.

In some aspect, the clarification is performed with a filter. In some aspects, the filter has pore sizes spanning between about 0.1 μm to about 1.5 μm. In some aspects, the filter has pore sizes spanning between about 0.2 μm to about 1.5 μm. In some aspects, the filter has pore sizes spanning between about 0.45 μm to about 0.8 μm. In some aspects, the filter has pore sizes spanning between about 0.45 μm to about 1.5 μm. In some aspects, the filter has a pore size of about 0.1 μm, about 0.2 μm, about 0.22 μm, about 0.45 μm, about 0.65 μm, about 0.8 μm, about 1.0 μm, about 1.2 μm, about 1.3 μm, or about 1.5 μm. In some aspects, the maximum pore size is 0.1 μm, 0.2 μm, 0.22 μm, 0.45 μm, 0.65 μm, 0.8 μm, 1.0 μm, 1.2 μm, 1.3 μm, or 1.5 μm.

In some aspects, the clarified supernatant is treated with a nuclease to degrade contaminating nucleic acid such as DNA and/or RNA, particularly from the producer cells. In some aspects, the nuclease is a DNAse or an RNAse. In some aspects, the nuclease is both a DNAse and an RNAse.

In some aspects, the improved end product of the present disclosure comprises an undetectable amount of the nuclease(s). Any suitable nuclease can be used in the methods described herein. In some aspects the nuclease is BENZONASE nuclease (EP 0229866 and U.S. Pat. No. 5,173,418), which degrades all forms of DNA and RNA, including single stranded, double stranded, linear, and circular). BENZONASE nuclease can be commercially obtained from Merck KGaA. BENZONASE is a genetically engineered endonuclease from Serratia marcescens. The protein is a dimer of 30 kDa subunits with two essential disulfide bods. This endonuclease attacks and degrades all forms of DNA and RNA (single stranded, double stranded, linear, and circular) and is effective over a wide range of operating conditions. BENZONASE possesses both DNAse and RNAse activity and no proteolytic activity.

In some aspects, the nuclease is DENARASE nuclease. DENARASE nuclease can be commercially obtained from c-LEcta GmbH. In some aspects, the nuclease is a DNase and/or RNase commonly used within the art for the purpose of eliminating unwanted or contaminating DNA and/or RNA from a preparation. DENARASE is described in US2012/0135498, including as sequence identifier number 3 therein. DENARASE is a genetically engineered endonuclease from Serratia marcescens. DENARASE possesses both DNAse and RNAse activity and no proteolytic activity.

In some aspects, the purification protocol comprises more than one nuclease treatment. In some aspects, each nuclease treatment comprises only a single type of nuclease, for example, only DENARASE. In some aspects, each nuclease treatment can comprise one or more nuclease. In some aspects, the first nuclease treatment comprise only one type of nuclease and the second nuclease treatment comprises one or more different types of nucleases. In some aspects, the first nuclease treatment comprises one or more different types of nucleases and the second nuclease treatment comprises only one type of nuclease.

In some aspects, the one or more nucleases is an endonuclease. In some aspects, the one or more nucleases is an exonuclease. In some aspects, the one or more nucleases comprise both an endonuclease and an exonuclease. In some aspects, the endonuclease or exonuclease is a modified enzyme isolated from a bacterium or a fungus. In some aspects, the endonuclease or exonuclease is a genetically modified enzyme that possesses still possesses endonuclease and/or exonuclease activity.

In some aspects, one or more of the viral purification processes described herein are performed after one or more nuclease treatments. In some aspects, the one or more nuclease treatments are performed post-treatment, prior to the final ultrafiltration/diafiltration process. In some aspects, one or more of the viral purification processes described herein are performed in place of nuclease treatment. In some aspects, the nuclease treatment is omitted from the viral purification process.

In some aspects, the nuclease treatment step is performed at about 1° C., about 2° C., about 3° C., about 4° C., about 5° C., about 6° C., about 7° C., about 8° C., about 9° C., about 10° C., about 11° C., about 12° C., about 13° C., about 14° C., or about 15° C. In some aspects, a nuclease treatment immediately precedes or immediately follows the clarification step. In some aspects, a nuclease treatment is the penultimate step of the purification protocol.

In some aspects, the dilution step dilutes the sample of lentivirus vectors to about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, or about 9×10⁷ infectious titer units per ml. In some aspects, the dilution step dilutes the sample of lentivirus vectors to a maximum of 5×10⁶ infectious titer units per ml.

In some aspects, diluting the amount of retroviral (e.g., lentiviral) vectors prior to one or more of the nuclease treatment steps results in an increase in the retroviral (e.g., lentiviral) vector titer in the final product. In some aspects, this increase is an increase of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 100%, at least 1 about 25%, at least about 150%, at least about 175%, at least about 200%, at least about 250%, at least about 300%, at least about 350%, or at least about or up to about 400%, relative to a control purification assay where the retroviral (e.g., lentiviral) vector titer was not diluted.

In some aspects, the methods described herein for purification of retroviral (e.g., lentiviral) vectors result in the removal of impurities left over from (1) plasmids used in producing the retroviral vectors, and/or (2) cells used in producing the retroviral vectors. In some aspects, the impurities are cellular debris, residual cellular DNA, and/or residual plasmid DNA. In some aspects, a measure of the presence of VSV-G provides an indication of the presence of residual plasmid DNA.

In some aspects, the methods described herein produce an improved end product, which is a purified sample of retroviral (e.g., lentiviral) vectors. In some aspects, this improvement is the removal of residual cellular DNA from the cells used to produce the vectors. In some aspects, this improvement is the removal of residual plasmid DNA used to shuttle the retroviral nucleic acids into cells used to produce the vectors. In some aspects, the residual cellular DNA is residual nuclear DNA.

In some aspects, the improved end product exhibits less than about 0.00001%, less than about 0.00005%, less than about 0.0001%, less than about 0.0005%, less than about 0.001%, less than about 0.005%, less than about 0.01%, less than about 0.05%, less than about 0.1%, less than about 0.5%, or less than about 1% of the residual cellular DNA as compared to the amount of cellular DNA present in the sample after clarification.

In some aspects, the improved end product exhibits less than about 0.00001%, less than about 0.00005%, less than about 0.0001%, less than about 0.0005%, less than about 0.001%, less than about 0.005%, less than about 0.01%, less than about 0.05%, less than about 0.1%, less than about 0.5%, or less than about 1% of the residual plasmid DNA as compared to the amount of plasmid DNA present in the sample after clarification.

In some aspects, the improved end product exhibits less than about 0.00001%, less than about 0.00005%, less than about 0.0001%, less than about 0.0005%, less than about 0.001%, less than about 0.005%, less than about 0.01%, less than about 0.05%, less than about 0.1%, less than about 0.5%, or less than about 1% of total residual DNA as compared to the amount of residual DNA present in the sample after clarification.

In some aspects, after clarification, the vector suspension is subjected to a concentration step via ultrafiltration/diafiltration. In some aspects, ultrafiltration/diafiltration may occur once during the purification process. In some aspects, ultrafiltration/diafiltration may occur more than once during the purification process. Ultrafiltration/diafiltration is used to concentrate the vector by forcing diluent to be passed through a filter in such a manner that the diluent is removed from the vector preparation whereas the vector is unable to pass through the filter and thereby remains, in concentrated form, in the vector preparation. In some aspects, the ultrafiltration/diafiltration process is tangential flow filtration (TFF) as described in, e.g., MILLIPORE catalogue entitled “Pharmaceutical Process Filtration Catalogue” pp. 177-202 (Bedford, Massachusetts, 1995/96). TFF is widely used in the bioprocessing industry for cell harvesting, clarification, purification and concentration of products including viruses. The system is composed of three distinct process streams: the feed solution, the permeate, and the retentate. Depending on application, filters with different pore sizes may be used. In the present invention the retentate contains the product (retroviral or lentiviral vector). Here to, the particular ultrafiltration membrane selected will have a pore size sufficiently small to retain the vector but large enough to effectively clear impurities. Depending on the manufacturer and membrane type, for retroviral vectors nominal molecular weight cutoffs (NMWC) between 100 and 1000 kDa may be appropriate, for instance membranes with 300 kDa or 500 kDa NMWC. In preferred aspects, a hollow fiber is used for the first or all TFF steps in the process. In some aspects, a hollow fiber is used for all TFF steps. In some aspects, the molecular weight cutoff is between about 10 to about 1,000 kDa. In some aspects, the molecular weight cutoff is between about 10 kDa to about 750 kDa, about 10 to about 500 kDa, about 10 kDa to about 250 kDa, about 10 kDa to about 100 kDa, about 100 kDa to about 1,000 kDa, about 100 kDa to about 750 kDa, about 100 kDa to about 500 kDa, about 100 kDa to about 250 kDa, about 250 kDa to about 1,000 kDa, about 250 kDa to about 750 kDa, about 250 kDa to about 500 kDa, about 500 kDa to about 1,000 kDa, about 500 kDa to about 750 kDa, or about 750 kDa to about 1,000 kDa. In preferred aspects, the hollow fiber is a 750 kDa hollow fiber.

In some aspects, the vectors are purified via a high-speed centrifugation process to concentrate the vector. In some aspects, the high-speed centrifugation is at least greater than 10,000×g. In some aspects, ultracentrifugation is utilized to purify the vector. In some aspects, the vector is purified through a gradient preparation, such as sucrose, iodixanol, etc. In some aspects, the vector is subjected to one or more solutions or compounds capable of concentrating the vector, such as polyethylene glycol (PEG) or LENTI-X CONCENTRATOR. In some aspects, the vector subjected to the one or more concentrating solutions or compounds is centrifuged to pellet the vector. In some aspects, the pellet is resuspended in a buffer, thus producing a vector suspension.

In some aspects, the viral vector purification process comprises a column purification step. The column purification step may comprise column chromatography. This step, as known in the art, separates the vector particles from cellular debris and other contaminants for the further purification of the viral vector particles. In some aspects, this step may be an ion exchange column purification, e.g., anion exchange or cation exchange. In some aspects, the column chromatography can be size exclusion chromatography. In some aspects, the column chromatography can be affinity chromatography. In some aspects, the column chromatography can be immobilized metal ion affinity chromatography. In some aspects, the column chromatography can comprise more than one chromatography strategy. In some aspects, the chromatography is performed in an open chromatography system. In some aspects, the chromatography is performed in a closed chromatography system.

In some aspects, the methods of the present disclosure do not utilize ion exchange chromatography. In some aspects, the methods of the present disclosure do not utilize anion exchange chromatography. In, some aspects, the methods of the present disclosure drawn to purifying a lentiviral vector do not utilize ion or anion exchange chromatography.

Proceeding any or all of the steps as described above, the vector is filter sterilized. Filter sterilization is a common process for pharmaceutical grade materials, and is known to one skilled in the art. Filter sterilization removes remaining contaminants in the viral vector preparation. The level of contaminants following filter sterilization should be so that the vector preparation is clinical use. In some aspects, filter sterilization is performed in aseptic conditions. Suitable filters are well known to one skilled in the art. In some aspects, the sterilizing filter has a pore size of 0.22 μm.

In some aspects, a lentiviral vector preparation is purified via chromatography after filter sterilization but prior to ultrafiltration/diafiltration. To reduce potential contamination events, the chromatography is preferably performed using a closed system process. In some aspects, the chromatography is selected from ion exchange chromatography, multimodal chromatography, size exclusion chromatography, size exclusion chromatography, and affinity chromatography. In some aspects, the chromatography may be repeated two or more times with the same type of chromatography for each repeat or a different type of chromatography for at least one of the repeats.

### V. Target Cells

In some aspects, the viral vector of the present invention may be used to modify a target cell. In some aspects, the viral vector may introduce a transgene or a nucleic acid sequence of interest to an immune cell or population of immune cells. In some aspects, the transgene or a nucleic acid sequence of interest encodes an exogenous antigen receptor. In some aspects, the exogenous antigen receptor is a chimeric antigen receptor (CAR) or a T cell receptor (TCR). In some aspects, the cell is a mammalian cell. In some aspects, the mammalian cell may be an immune cell or precursor cell thereof. In some aspects, the immune cell or precursor cell thereof may be a T cell. In some aspects, the T cell can be a cytotoxic T cell, a regulatory T cell, NKT cells. In exemplary aspects, the T cell is a CD8+ T cell and/or a CD4+ T cell.

In some aspects, the immune cell or a population of immune cells are harvested from an apheresis sample from a patient. In some aspects, the apheresis sample is cryopreserved prior to harvesting of the immune cell or population of immune cells. In some aspects, the apheresis sample is a fresh apheresis sample from a patient that has not been cryopreserved. In some aspects, the immune cell or population of immune cells are obtained from an apheresis sample during a process or protocol which comprises an enrichment step. In some aspects, the modified cell is an autologous cell. In some aspects, the modified cell is an allogeneic cell.

In some aspects, the immune cell or population of immune cells modified by the viral vector of the present invention may be used to treat, prevent, or ameliorate a disease or disorder. In some aspects, the disease or disorder may include, but is not limited to, malignancy disorders including cancer, benign and malignant tumor growth, metastases, angiogenesis; autoimmune diseases, including arthritis, rheumatoid arthritis, allergic reactions, asthma, and systemic lupus erythematosus.

### VI. Pharmaceutical Compositions

Pharmaceutical compositions of the present invention may comprise a genetically modified immune cell as described herein, in combination with one or more pharmaceutically or physiologically acceptably carriers, diluents, adjuvants, or excipients. Such compositions may comprise buffers such as neutral buffered saline, phosphate buffered saline and the like; carbohydrates such as glucose, mannose, sucrose, or dextrans, mannitol; proteins; polypeptides or amino acids such as glycine, antioxidants; chelating agents such as EDTA or glutathione; adjuvants (e.g., aluminum hydroxide); and preservatives. In some aspects, compositions of the present invention are preferably formulated for intravenous administration. In some aspects, the pharmaceutical compositions comprising the retroviral (e.g., lentiviral) vectors are suitable for administering to a patient. In some aspects, the pharmaceutical compositions comprising the retroviral (e.g., lentiviral) vectors are suitable for administering to cells, which are administered to a patient.

### VII. Definitions

While the following terms are believed to be well understood by one of ordinary skill in the art, the following definitions are set forth to facilitate explanation of the presently disclosed subject matter.

The term “a” or “an” may refer to one or more of that entity, i.e. can refer to plural referents. As such, the terms “a” or “an”, “one or more” and “at least one” are used interchangeably herein. In addition, reference to “an element” by the indefinite article “a” or “an” does not exclude the possibility that more than one of the elements is present, unless the context clearly requires that there is one and only one of the elements.

Reference throughout this specification to “one embodiment”, “an embodiment”, “one aspect”, or “an aspect” means that a particular feature, structure or characteristic described in connection with the embodiment is included in at least one embodiment of the present disclosure. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments.

As used herein, the terms “about” or “approximately” when preceding a numerical value indicates the value plus or minus a range of 10% of the value.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various aspects of the disclosure can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, a “control” is an alternative sample used in an experiment for comparison purpose. A control can be “positive” or “negative.” A “control sample” or “reference sample” as used herein, refer to a sample or reference that acts as a control for comparison to an experimental sample. For example, an experimental sample comprises compound A, B, and C in a vial, and the control may be the same type of sample treated identically to the experimental sample, but lacking one or more of compounds A, B, or C.

The present disclosure provides novel methods and compositions for the manufacture and purification of viral vectors. In certain aspects, a process for producing a lentiviral vector formulation as provided herein, comprises treating a filter-sterilized lentiviral vector preparation with a nuclease, and concentrating the nuclease-treated lentiviral vector preparation to produce the lentiviral vector formulation, wherein the step of concentrating is the final step in the process. In certain aspects, a process for producing a lentiviral vector formulation, as provided herein, comprises in chronological order: (i) culturing cells that produce the lentiviral vector; (ii) collecting the supernatant comprising the lentiviral vector; (iii) clarifying the supernatant; (iv) concentrating the clarified supernatant, comprising exchange of the concentrated supernatant with formulation buffer; (v) purifying the lentiviral vector from the concentrated supernatant to produce a lentiviral vector preparation; (vii) treating the filter-sterilized lentiviral vector preparation with nuclease; and (viii) concentrating the nuclease-treated lentiviral vector preparation to produce a final product.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by M R Green and J. Sambrook and Harlow et al., Antibodies: A Laboratory Manual, Chapter 14, Cold Spring Harbor Laboratory, Cold Spring Harbor (2013, 2nd edition).

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. Polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, the term “composition” is intended to encompass a product containing the specified ingredients (e.g., a viral vector as provided herein) in, optionally, the specified amounts, as well as any product which results, directly or indirectly, from combination of the specified ingredients in, optionally, the specified amounts.

As used herein, the term “vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai virus vectors, adenovirus vectors, adeno-associated virus vectors, retrovirus vectors, lentivirus vectors, and the like.

As used herein, the term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

As used herein, the term “expression” is defined as the transcription and/or translation of a particular nucleotide sequence driven by its promoter.

As used herein, the term “lentivirus” refers to a genus of the Retroviridae family. Lentiviruses are unique among the retroviruses in being able to infect non-dividing cells. They can deliver a significant amount of genetic information into the DNA of the host cell, so they are one of the most efficient methods of gene delivery. HIV, SIV, and FIV are all examples of lentiviruses. Vectors derived from lentiviruses offer the means to achieve significant levels of gene transfer in vivo.

As used herein, the term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

As used herein, the term “residual nucleic acids” refers to the non-retroviral (e.g., non-lentiviral) nucleic acids present in the samples of the present disclosure after clarification of the retroviral (e.g., lentiviral) vectors. Similarly, the term “residual DNA” refers to the non-retroviral (e.g., non-lentiviral) nucleic acids present in the samples of the present disclosure after clarification of the retroviral (e.g., lentiviral) vectors. The residual nucleic acids refer to DNA and RNA. The residual nucleic acids refer to the genetic material corresponding to the nuclear DNA and/or the plasmids remaining in the samples described herein after clarification of the vectors.

Unless otherwise specified, a “nucleotide sequence encoding an amino acid sequence” includes all nucleotide sequences that are degenerate versions of each other and that encode the same amino acid sequence. The phrase “nucleotide sequence that encodes a protein or an RNA” may also include introns to the extent that the nucleotide sequence encoding the protein may in some version contain an intron(s).

The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular aspects only, and is not intended to be limiting.

### EXAMPLES

### Example 1

### Retroviral Vector Manufacturing and Purification

HEK293T cells were expanded in tissue culture flasks, and then into 10-layer cell factories for up to 14 days. On day 14, the cells were transfected with plasmids encoding a third-generation lentiviral vector system, and culture media containing the viral vectors was harvested two days later, on day 16.

The cell culture media was clarified. After clarification and prior to the first tangential flow filtration (TFF), the clarified media was treated with DENARASE nuclease (c-LEcta GmbH; Leipzig, Germany) to remove residual cellular genomic DNA and to prevent clogging of the filter. The vector-containing nuclease-treated clarified media was then concentrated in an ultrafiltration/diafiltration step using TFF in a formulation buffer (Tris, salt, sugar, and approximately neutral pH). The TFF was a 750 kDa hollow fiber column. The formulation buffer comprising the vector was then passed through a column of CAPTOCORE 700 resin (GE) to remove host cell proteins, serum proteins, nucleic acids, and residual nuclease. The resulting preparation was then diluted, filter sterilized, and subsequently treated with DENARASE nuclease a second time. This again removed any remaining contaminating DNA effectively. The vector preparation was then passed through a 750 kDa hollow fiber once more as a final ultrafiltration step to remove the nuclease to produce a final product. The process is illustrated by FIG. 1.

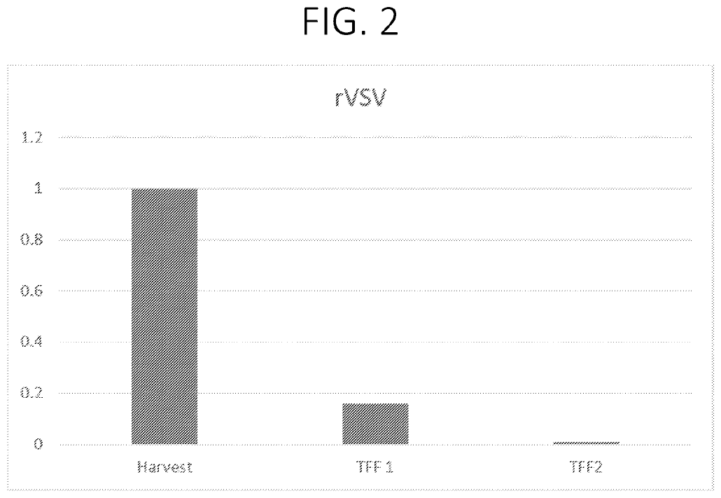

To track the removal of residual DNA throughout the process, the presence of the vesicular stomatitis virus glycoprotein gene (VSV-G) was detected using quantitative PCR (qPCR) at different steps of the process. Samples were taken after clarification of the cell culture supernatant, after the first TFF step, and then of the final product. FIG. 2 illustrates the decrease in the residual DNA from the harvesting of the vectors to an almost undetectable level by the end of the process in the final product.

In particular, FIG. 2 illustrates the results of a qPCR assay in which a vesicular stomatitis virus glycoprotein (VSV-G) gene was detected and quantified. The VSV-G detection effectively measures the amount of residual VSV-G still present at multiple points in the vector manufacturing process. The amount of residual VSV-G is generally a measure for the amount of residual plasmid contaminants left over from production of the vector. Table 1 below identifies the step removal percent seen after three steps in the process of producing the vectors. FIG. 2 is a graphical representation of the “rVSV DNA Remaining” data presented in Table 1. The “Clarified harvest” sample was collected immediately after clarification. The “TFF 1” sample was collected immediately after the first tangential flow filtration step. The “Final Product” sample was collected immediately after the final tangential flow filtration step, thus yielding the end result of the lentiviral vector production and purification method.

TABLE 1
Minimizing DNA impurities from lentiviral plasmids and host cells

| | rVSV DNA | rHost Cell |
|---|---|---|
| Sample | Remaining | DNA Remaining |
| Clarified harvest | 1 | 1 |
| TFF 1 | 0.16 | 0.017 |
| Final Product | 0.009 | 0.0003 |

With a second nuclease step as the penultimate step of the process, there was a question regarding how much residual nuclease would remain in the formulation after the TFF steps, and then ultimately in the final product. The detection and quantification of residual endonuclease is performed using the MIILLIPORESIGMA BENZONASE ELISA Kit II, #1016810001, which is an enzyme-linked immunosorbent (ELISA) assay with antibodies specific for BENZONASE endonuclease. During the evaluation of the BENZONASE ELISA Kit II, the assay was tested using a GMP-grade sample of DENARASE. For this evaluation, multiple dilutions of DENARASE were measured using the BENZONASE-specific antibodies and the detection of DENARASE was confirmed. Table 2 demonstrates that the level of residual DENARASE nuclease after the TFF steps was nearly below the limit of detection, indicating that the final product comprises a miniscule amount of residual endonuclease.

TABLE 2
Residual DENARASE assay results

| Sample | Residual DENARASE (ng/ml) |
|---|---|
| Final TFF | <1 |

### Example 2

### Lentiviral Titers

Lentiviral vector titration was performed using the human T cell lymphoblastic lymphoma cell line SupT1 and the percentage of SupT1 cells expressing the transgene was determined by flow cytometry.

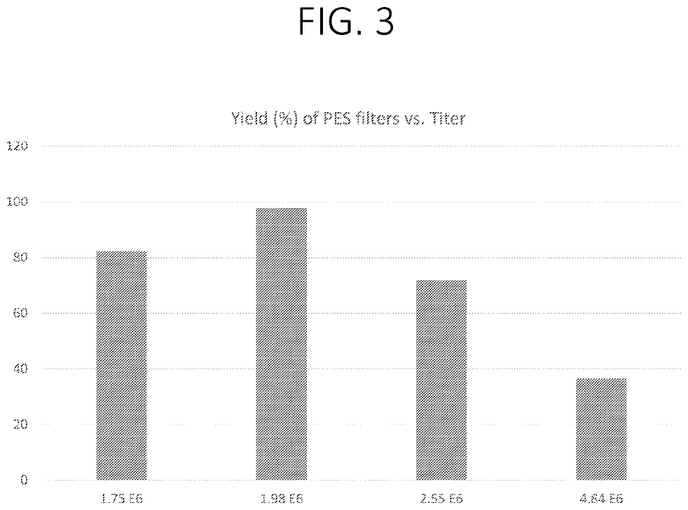

Four samples were evaluated for the quantity of lentivirus in each sample (SupT1 cells by flow cytometry. The samples were clarified, filter sterilized, and subjected to the methods described herein for lentiviral isolation and purification. For each sample, the methods were varied by the amount the lentivirus was diluted prior to being subjected to sterile filtration, such that the titers spanned 1.75×10⁶ to 4.84×10⁶. FIG. 3 presents each of the lentiviral titers and their corresponding final yield. Based upon FIG. 3, it is apparent that a standard dose (titer) dependent curve between titer and percent yield does not exist. Instead, the optimal titer appears to be 2×10⁶, with a slight decrease in the final yield at a titer of 1.75×10⁶. However, marked decreases in the final yield of the lentiviral vectors occurred at higher titers (e.g., 2.55×10⁶ and 4.84×10⁶, as depicted in FIG. 3.

### Example 3

### Residual DNA Assay

Detection and quantitation of residual vesicular stomatitis virus glycoprotein gene (VSV-G) was performed using a Quantitative PCR (QPCR) assay with primers and probe specifically targeting the VSV-G. Results shown in Table 3 are a summary of three large scale runs.

Residual DENARASE assay: The detection and quantification of residual endonuclease was performed using the Millipore Sigma BENZONASE ELISA Kit II, #1016810001, which is an enzyme-linked immunosorbent (ELISA) assay with antibodies specific for BENZONASE Endonuclease. During the evaluation of the BENZONASE ELISA Kit II, the assay was tested using a GMP-grade sample of DENARASE. For this evaluation, multiple dilutions of DENARASE were measured using the BENZONASE-specific antibodies and the detection of DENARASE was confirmed. The results of residual DENARASE are shown in Table 3:

TABLE 3

| | TFF 1 rVSV- | TFF2 r VSV- | rDENARASE |
|---|---|---|---|
| | g Left (%) | g left % | (ng/ml) |
| Run 1 | 8.1 | 0.14 | 1.73 |
| Run 2 | 16.6 | 0.09 | 1.34 |
| Run 3 | 10.2 | 0.2 | 1.40 |

### Example 4

### Scaled Production

The methods of producing clinical grade lentiviral vectors described herein have been performed at clinical scale under Good Manufacturing Practice (GMP) conditions as promulgated by the United States Food and Drug Administration. The clinical scale is defined as 20-60 patient doses per batch at between 7×10⁶ TU/ml to 8×10⁷ TU/ml, where TU is transduction units.

The methods illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof. It is recognized that various modifications are possible within the scope of the disclosure claimed. Thus, it should be understood that although the present disclosure has been specifically disclosed by preferred aspects and optional features, modification and variation of the disclosure embodied therein herein disclosed may be resorted to by those skilled in the art, and that such modifications and variations are considered to be within the scope of this disclosure.

The disclosure has been described broadly and generically herein. Each of the narrower species and subgeneric groupings falling within the generic disclosure also form part of the methods. This includes the generic description of the methods with a proviso or negative limitation removing any subject matter from the genus, regardless of whether or not the excised material is specifically recited herein. The present technology is not to be limited in terms of the particular aspects described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular aspects only, and is not intended to be limiting.

One skilled in the art readily appreciates that the present disclosure is well adapted to carry out the objects and obtain the ends and advantages mentioned, as well as those inherent therein. Modifications therein and other uses will occur to those skilled in the art. These modifications are encompassed within the spirit of the disclosure and are defined by the scope of the claims, which set forth non-limiting aspects of the disclosure.

In addition, where features or aspects of the disclosure are described in terms of Markush groups, those skilled in the art will recognize that the disclosure is also thereby described in terms of any individual member or subgroup of members of the Markush group.

All references, articles, publications, patents, patent publications, and patent applications cited herein are incorporated by reference in their entireties for all purposes.

However, mention of any reference, article, publication, patent, patent publication, and patent application cited herein is not, and should not be taken as, an acknowledgment or any form of suggestion that they constitute valid prior art or form part of the common general knowledge in any country in the world.

1. A process for producing a lentiviral vector formulation, the process comprising:
(a) treating a filter-sterilized lentiviral vector preparation with a nuclease, and
(b) concentrating the nuclease-treated lentiviral vector preparation to produce the lentiviral vector formulation;
wherein the step of concentrating is the final step in the process.

2. The process according to claim 1, wherein the process comprises a clarification of cell culture supernatant.

3. The process according to claim 2, wherein the clarification is followed by a first nuclease treatment.

4. The process according to claim 3, wherein the nuclease possesses endonuclease activity.

5. The process according to claim 3, wherein the nuclease possesses exonuclease activity.

6. The process according to claim 4, wherein the nuclease is a modified nuclease from Serratia marcescens.

7. The process according to claim 3, wherein the first nuclease treatment precedes an ultrafiltration/diafiltration step.

8. The process according to claim 7, wherein the ultrafiltration/diafiltration step comprises tangential flow filtration.

9. The process according to claim 7, wherein the ultrafiltration/diafiltration step comprises hollow fiber filtration.

10. The process according to claim 1, wherein the lentiviral vector preparation is diluted prior to the filter-sterilization of the lentiviral vector preparation.

11. The process according to claim 10, wherein the lentiviral vector preparation is diluted into a formulation buffer.

12. The process according to claim 11, wherein the lentiviral vector preparation is diluted into the formulation buffer during the ultrafiltration/diafiltration step.

13. The process according to claim 1, wherein the process comprises a second nuclease treatment.

14. The process according to claim 13, wherein the second nuclease treatment is the penultimate step in the process.

15. The process according to claim 14, wherein the nuclease possesses endonuclease activity.

16. The process according to claim 14, wherein the nuclease possesses exonuclease activity.

17. The process according to claim 15, wherein the nuclease is a modified nuclease from Serratia marcescens.

18. A process for producing a lentiviral vector formulation, the process comprising:
(a) culturing cells that produce the lentiviral vector;
(b) collecting supernatant from the cultured cells;
(c) clarifying the supernatant;
(d) concentrating the clarified supernatant;
(e) purifying the lentiviral vector from the concentrated supernatant to produce a lentiviral vector preparation;
(f) filter-sterilizing the lentiviral vector preparation;
(g) treating the filter-sterilized lentiviral vector preparation with one or more nucleases; and
(h) concentrating the nuclease-treated lentiviral vector preparation to produce a final product.

19. A process for producing a lentiviral vector formulation, the process comprising:
(a) culturing cells that produce the lentiviral vector;
(b) collecting supernatant from the cultured cells;
(c) clarifying the supernatant;
(d) concentrating the clarified supernatant
(e) purifying the lentiviral vector from the concentrated supernatant to produce a lentiviral vector preparation;
(f) filter-sterilizing the lentiviral vector preparation; and
(g) concentrating the nuclease-treated lentiviral vector preparation to produce a final product;
wherein the clarified supernatant and/or the lentiviral vector preparation are treated with a nuclease.

20. The process according to claim 18, wherein concentrating the clarified supernatant comprises exchanging the concentrated supernatant with a formulation buffer.

21. The process according to claim 19, wherein the clarified supernatant and the lentiviral vector preparation are treated with a nuclease.

22. The process according to claim 19, wherein the clarified supernatant is treated with the nuclease prior to (d),

23. The process according to claim 19, wherein the clarified supernatant is treated with the nuclease after (c).

24. The process according to claim 18, wherein the nuclease possesses endonuclease activity.

25. The process according to claim 18, wherein the nuclease possesses exonuclease activity.

26. The process according to claim 24, wherein the nuclease is a modified nuclease from Serratia marcescens.

27. A process for producing a lentiviral vector formulation, comprising in chronological order:
(a) culturing cells that produce the lentiviral vector;
(b) collecting the supernatant comprising the lentiviral vector;
(c) clarifying the supernatant;
(d) treating the clarified supernatant with a nuclease;
(e) concentrating the nuclease-treated clarified supernatant, comprising exchange of the concentrated supernatant with formulation buffer;
(f) purifying the lentiviral vector from the concentrated supernatant to produce a lentiviral preparation;
(g) filter-sterilizing the lentiviral vector preparation;
(h) treating the filter-sterilized lentiviral vector preparation with a nuclease; and
(i) concentrating the nuclease-treated lentiviral vector preparation to produce a final product.
